(19) United States
(12) Patent Application Publication
(10) Pub. No.: US 2018/0325953 A1
(43) Pub. Date: Nov. 15, 2018

(54) MODIFIED IMMUNE CELLS AND USES THEREOF

(71) Applicants: APERISYS, INC., Destin, FL (US); THE GENERAL HOSPITAL CORPORATION (DBA MASSACHUSETTS GENERAL HOSPITAL), Boston, MA (US)

(72) Inventors: Mark C. Poznansky, Destin, FL (US); David Peritt, Destin, FL (US); Patrick Reeves, Destin, FL (US)

(21) Appl. No.: 15/774,878

(22) PCT Filed: Nov. 9, 2016
(86) PCT No.: PCT/US16/61207

§ 371 Date: May 9, 2018

Related U.S. Application Data
(60) Provisional application No. 62/253,021, filed on Nov. 9, 2015.
(60) Provisional application No. 62/253,072, filed on Nov. 9, 2015.
(60) Provisional application No. 62/253,093, filed on Nov. 9, 2015.
(60) Provisional application No. 62/253,096, filed on Nov. 9, 2015.
(60) Provisional application No. 62/327,877, filed on Apr. 26, 2016.

(51) Int. Cl. A61K 35/17; C07K 14/725; A61P 35/00; C07K 14/715; C12N 5/0783; A61K 45/06; A61K 39/00

(52) U.S. Cl. CPC A61K 35/17; C07K 14/7051; A61P 35/00; A61K 39/001121; C12N 5/0636; A61K 45/06; C07K 14/7158

(57) ABSTRACT

The invention described herein relates to methods and compositions for treating cancer in a patient by administering an effective amount of cytokine receptor modified immune cells.

### CROSS-REFERENCE TO RELATED APPLICATIONS

This application claims priority under 35 U.S.C. § 119(e) to U.S. Provisional Application Nos. 62/253,093, filed Nov. 9, 2015; 62/327,877, filed Apr. 26, 2016; 62/253,072, filed Nov. 9, 2015; 62/253,096, filed Nov. 9, 2015; and 62/253,021, filed Nov. 9, 2015; each of which is incorporated herein by reference in its entirety.

### BACKGROUND OF THE INVENTION

Cell movement in response to specific stimuli is observed in prokaryotes and eukaryotes. Cell movement seen in these organisms has been classified into three types: chemotaxis or the movement of cells along a gradient towards an increasing concentration of a chemical; negative chemotaxis which has been defined as the movement down a gradient of a chemical stimulus; and chemokinesis or the increased random movement of cells induced by a chemical agent.

Embodiments described herein relate generally to technology and subject matter related to treatments and compositions that can modify movement of cells, for example in relation to the treatment of cancer and tumors. For example, the embodiments relate to technology that can target tumors to effectively and efficiently kill tumors and/or metastasizing cancer cells.

### SUMMARY OF THE INVENTION

Chemotaxis and chemokinesis occur in mammalian cells in response to the class of proteins, called chemokines. Additionally, chemorepellent, or fugetactic, activity has been observed in mammalian cells. For example, some tumor cells secrete concentrations of chemokines that are sufficient to repel immune cells from the site of a tumor, thereby reducing the immune system's ability to target and eradicate the tumor. Metastasizing cancer cells may use a similar mechanism to evade the immune system. Repulsion of immune cells, such as tumor antigen-specific T-cells, e.g. from a tumor expressing high levels of CXCL12 or interleukin 8 (IL-8), allows the tumor cells to evade immune control.

CXCR7 is a protein that in humans is encoded by the CXCR7 gene. CXCR7 receptors are expressed by a variety of cells, and have key functions in promoting tumor development and progression. CXCR7 is a chemokine receptor that is able to bind stromal-derived-factor-1 (SDF-1, also known as CXCL12), a molecule endowed with potent chemotactic activity for lymphocytes, and interferon-inducible T-cell alpha chemoattractant (I-TAC, also known as CXCL11). CXCL12 is known to be important in hematopoietic stem cell homing to the bone marrow and in hematopoietic stem cell quiescence. In addition, CXCR7 expression seems to be enhanced during pathological inflammation and tumor development. Reports suggest that CXCR7 may function, at least in part, as a decoy receptor, acting as a CXCL12 (and CXCL11) scavenger, with the ability to promote CXCL12 internalization and degradation.

CXCR4 is a protein that in humans is encoded by the CXCR4 gene. CXCR4 receptors are expressed by a variety of normal cells, including immune cells (e.g., T cells, B cells, and natural killer [NK] cells). CXCR4 is an alpha-chemokine receptor specific for CXCL12, a molecule endowed with potent chemotactic activity for lymphocytes. CXCL12, a ligand for CXCR4, is known to be important in hematopoietic stem cell homing to the bone marrow and in hematopoietic stem cell quiescence. While CXCR4 expression is low or absent in many healthy tissues, it is overexpressed in many types of cancer, including breast cancer, ovarian cancer, melanoma, and prostate cancer. Expression of this receptor in cancer cells has been linked to metastasis to tissues containing a high concentration of CXCL12, such as lungs, liver and bone marrow.

As many as 85% of solid tumors and leukemias express CXCL12 at a level sufficient to have fugetactic effects, e.g. repulsion of immune cells from the tumor, also referred to as the “fugetactic wall.”. Cancers that express CXCL12 at such levels include, but are not limited to, prostate cancer, lung cancer, breast cancer, pancreatic cancer, ovarian cancer, gastric cancer, esophageal cancer, and leukemia.

Accordingly, there remains a need for treatments and compositions that target tumors to effectively and efficiently kill tumors and/or metastasizing cancer cells.

This instant technology generally relates to immune cells overexpressing CXCR7 receptors, lacking CXCR4 receptors, or both overexpressing CXCR7 receptors and lacking CXCR4 receptors on their cell surface and uses thereof for treating cancer.

Repulsion of tumor antigen-specific T-cells, e.g. from a tumor expressing high levels of CXCL12 or interleukin 8 (IL-8), allows the tumor cells to evade immune control. Without being bound by theory, it is believed that the immune cells with increased numbers of CXCR7 receptors on their cell surface, when administered to a patient, will be able, at least in part, to act as a decoy to bind and degrade the CXCL12-induced fugetactic wall in order to allow immune cells to detect and destroy tumor cells. It is also believed that the immune cells with fewer or no CXCR4 receptors, when administered to a patient, will be able, at least in part, to evade the fugetactic wall created by some tumors in order to detect and destroy tumor cells

Although anti-fugetactic agents alone provide promising results for cancer treatment, it is contemplated that therapy with immune cells over-expressing CXCR7 receptors, lacking CXCR4 receptors, or both overexpressing CXCR7 receptors and lacking CXCR4 receptors as described herein, and optionally in combination with anti-fugetactic agents, will result in more efficient tumor targeting and improved patient outcomes. Without being bound by theory, it is believed that such methods are especially beneficial, by way of non-limiting example, if the tumor is large in size, there are multiple tumors in the patient, the patient's immune system is compromised, etc.

As many as 85% of solid tumors and leukemias express CXCL12 at a level sufficient to have fugetactic effects, e.g. repulsion of immune cells from the tumor. Cancers that express CXCL12 at such levels include, but are not limited to, prostate cancer, lung cancer, breast cancer, pancreatic cancer, ovarian cancer, gastric cancer, esophageal cancer, and leukemia.

One aspect of the invention relates to an ex vivo modified immune cell modified to overexpress CXCR7 receptors. In one embodiment, the invention relates to an ex vivo modified immune cell wherein the CXCR7 gene or gene transcript is edited such that CXCR7 receptor is over expressed on an outer cell surface of the immune cell.

One aspect of the invention relates to an ex vivo modified immune cell modified to have no or substantially no CXCR4 receptors on an outer cell surface of the modified immune cell. In one embodiment, the invention relates to an ex vivo modified immune cell wherein the immune cell comprises a direct or indirect suppression of the CXCR4 gene or gene transcript such that CXCR4 receptor expression on the outer cell surface of the cell is reduced or eliminated.

One aspect of the invention relates to an ex vivo modified immune cell modified to overexpress CXCR7 receptors and modified to have no or substantially no CXCR4 receptors on an outer cell surface of the modified immune cell. In one embodiment, the invention relates to an ex vivo modified immune cell wherein the CXCR7 gene or gene transcript is edited such that CXCR7 receptor is over expressed on an outer cell surface of the immune cell and wherein the immune cell comprises a direct or indirect suppression of the CXCR4 gene or gene transcript such that CXCR4 receptor expression on the outer cell surface of the cell is reduced or eliminated.

One aspect of the invention relates to an ex vivo population of modified immune cells wherein at least a portion of the modified immune cells overexpress CXCR7 receptors and have no or substantially no CXCR4 receptors on an cell outer surface of the modified immune cell.

In one embodiment, the CXCR7 receptors bind CXCL12 when delivered to a patient.

In one embodiment, the immune cell evades fugetactic activity of tumor cells when delivered to a patient.

In one embodiment, a source of the immune cell is autologous, allogeneic, or xenographic, or combinations thereof.

In one embodiment, the immune cell is obtained from a patient having a cancer.

In one embodiment, the immune cell is a T-cell, a B-cell, a NK cell, or any combination thereof.

In one embodiment, the immune cell is further modified to express a tumor cell homing receptor on the outer cell surface of the immune cell, for example, a chimeric antigen receptor (CAR), an Fc receptor, or combinations thereof. In other embodiments, the immune cell expresses an endogenous tumor cell homing receptor that is not CXCR4.

In one embodiment, the CAR targets a cancer-associated antigen, for example, α-folate receptor, CAIX, CD19, CD20, CD30, CD33, CEA, EGP-2, erb-B2, erb-B 2,3,4, FBP, GD2, GD3, Her2/neu, IL-13R-a2, k-light chain, LeY, MAGE-AL Mesothelin, and PSMA.

In one embodiment, the immune cell has 10% or more of the amount of CXCR7 receptors on the outer cell surface as compared to an average number of CXCR7 receptors on an unmodified immune cell.

In one embodiment, the immune cell has 50% or less of the amount of CXCR4 receptors on the outer cell surface as compared to average number of CXCR4 receptors on an unmodified immune cell.

One aspect of the invention relates to a modified immune cell population comprising an effective amount of the modified immune cells as described herein. In one embodiment, the immune cell population comprises T-cells, B-cells, NK cells, or any combination thereof.

One aspect of the invention relates to a pharmaceutical composition comprising an effective amount of the modified immune cells as described herein and one or more pharmaceutically acceptable excipients. In other aspects, the invention relates to a pharmaceutical composition comprising an effective amount of CXCR7-modified immune cells and/or an effective amount of CXCR4-modified immune cells and/or an effective amount of CXCR7- and CXCR4-modified immune cells and one or more pharmaceutically acceptable excipients.

In one embodiment, the composition further comprises an anti-fugetactic agent. In one embodiment, the anti-fugetactic agent is bound to one or more receptors on the immune cell surface.

One aspect of the invention relates to a method for treating a patient having a tumor which expresses CXCL12 wherein said patient is administered an effective amount of modified immune cells or compositions as described herein.

In one embodiment, the fugetactic activity of tumor cells in the patient is reduced or eliminated, at least with respect to the modified immune cells.

In one embodiment, the immune cells are administered systemically to the patient. In another embodiment, the immune cells are administered locally, for example, directly to the tumor or tumor microenvironment.

In one embodiment, the immune cells are administered in combination with an anti-fugetactic agent, for example, AMD3100 (1,1′-[1,4-phenylenebis (methylene)]-bis-1,4,8,11-tetraazacyclotetradecane, also known as mozobil/plerixafor) or derivative thereof, KRH-1636, T-20, T-22, T-140, TE-14011, T-14012, TN14003, TAK-779, AK602, SCH-351125, Tannic acid, NSC 651016, thalidomide, GF 109230X.

In one embodiment, the immune cells and anti-fugetactic agent are administered sequentially. In another embodiment, the immune cells and anti-fugetactic agent are administered simultaneously.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

Throughout this disclosure, various publications, patents and published patent specifications are referenced by an identifying citation. The disclosures of these publications, patents and published patent specifications are hereby incorporated by reference in their entirety into the present disclosure.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g. horse, cow, pig, goat, sheep). In especially preferred embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disease or disorder; and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, remission of the cancer, inhibition of metastasis of the tumor, reduction or elimination of at least one symptom of the cancer, and the like.

The term “administering” or “administration” of an agent, drug, or a natural killer cell to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

The term “simultaneous” therapeutic use refers to the administration of at least two active ingredients by the same route and at the same time or at substantially the same time.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect. For example, an effective amount of a modified immune cell overexpressing CXCR7 receptors may be an amount sufficient to bind and sequester CXCL12 such that the fugetactic wall is reduced or eliminated. In another example, an effective amount of a modified immune cell lacking CXCR4 receptors may be an amount sufficient to evade the fugetactic effect and detect and destroy a cancer cell or tumor. The therapeutically effective amount of the modified immune cell will vary depending on the tumor being treated and its severity as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

The term “kill” with respect to a cell/cell population is directed to include any type of manipulation that will lead to the death of that cell/cell population.

“Antibodies” as used herein include polyclonal, monoclonal, single chain, chimeric, humanized and human antibodies, prepared according to conventional methodology.

“Cytokine” is a generic term for non-antibody, soluble proteins which are released from one cell subpopulation and which act as intercellular mediators, for example, in the generation or regulation of an immune response. See Human Cytokines: Handbook for Basic & Clinical Research (Aggrawal, et al. eds., Blackwell Scientific, Boston, Mass. 1991) (which is hereby incorporated by reference in its entirety for all purposes).

“CXCR4/CXCL12 antagonist” refers to a compound that antagonizes CXCL12 binding to CXCR4 or otherwise reduces the fugetactic effect of CXCL12.

By “fugetactic activity” or “fugetactic effect” it is meant the ability of an agent to repel (or chemorepel) a eukaryotic cell with migratory capacity (i.e., a cell that can move away from a repellant stimulus). The term also refers to the chemorepellent effect of a chemokine secreted by a cell, e.g. a tumor cell. Usually, the fugetactic effect is present in an area around the cell wherein the concentration of the chemokine is sufficient to provide the fugetactic effect. Some chemokines, including interleukin 8 and CXCL12, may exert fugetactic activity at high concentrations (e.g., over about 100 nM), whereas lower concentrations exhibit no fugetactic effect and may even be chemoattractant.

The term “anti-fugetactic effect” refers to the effect of the anti-fugetactic agent to attenuate or eliminate the fugetactic effect of the chemokine.

“Immune cells” as used herein are cells of hematopoietic origin that are involved in the specific recognition of antigens. Immune cells include antigen presenting cells (APCs), such as dendritic cells or macrophages, B cells, T cells, natural killer cells, etc.

The term “anti-cancer therapy” as used herein refers to traditional cancer treatments, including chemotherapy and radiotherapy, as well as vaccine therapy.

As used herein “chimeric antigen receptors” or “CARs” refer to fusion proteins comprised of an antigen recognition moiety and T-cell activation domains. Eshhar et al., (1993) Proc. Natl. Acad. Sci., 90(2): 720-724. A CAR is an artificially constructed hybrid protein or polypeptide containing an antigen binding domain of an antibody (e.g., a single chain variable fragment (scFv)) linked to T-cell signaling or T-cell activation domains. CARs have the ability to redirect T-cell specificity and reactivity toward a selected target (i.e., a tumor cell) in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

As used herein, the term “knockdown” refers to the reduction in the expression level of a protein in a cell. Accordingly, “knockdown” may be used interchangeably with the phrases “reduction of the levels of the protein,” “reduction in the expression level of a protein,” “reduction of the intracellular expression level of a protein” or any variation of these phrases.

As used herein, the term “knockout” refers to an in vitro engineered disruption of native chromosomal DNA, typically within a protein coding region, such that a foreign piece of DNA conveniently but not necessarily providing a dominant selectable marker is inserted within the native sequence or a piece of native chromosomal DNA is removed. A knockout mutation within a protein coding region prevents expression of the wild-type protein, which usually leads to loss of the function provided by the protein. The alteration may be an insertion, deletion, frameshift mutation, or missense mutation. Preferably, the alteration is an insertion or deletion, or is a frameshift mutation that creates a stop codon.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein (e.g., a CAR). The expression product itself, e.g. the resulting protein, may also be said to be “expressed”. An expression product can be characterized as intracellular, extracellular or secreted. The term “intracellular” means something that is inside a cell. The term “extracellular” means something that is outside a cell, e.g., on a cell surface. A substance is “secreted” by a cell if it appears in significant measure outside the cell, from somewhere on or inside the cell.

The term “overexpression,” as used herein, refers to increased expression of a gene and/or its encoded protein in a cell, such as an immune cell. A modified immune cell that “overexpresses” a protein is one that has higher levels of that protein compared to a unmodified immune cell of the same type, for example, about 10%, about 20%, about 30%, about 40%, about 50%, about 60%, about 70%, about 80%, about 90%, about 100%, about 200%, about 300%, or more expression of a protein.

The term “genetically modified” is meant to refer to a cell containing a gene that is altered from its native state (e.g. by insertion mutation, deletion mutation, nucleic acid sequence mutation, or other mutation), or that a gene product is altered from its natural state (e.g. by delivery of a transgene that works in trans on a gene's encoded mRNA or protein, such as delivery of inhibitory RNA or delivery of a dominant negative transgene).

The term “insertional mutation” is used herein to refer the translocation of nucleic acid from one location to another location which is in the genome of an animal so that it is integrated into the genome, thereby creating a mutation in the genome. Insertional mutations can also include knocking out or knocking in of endogenous or exogenous DNA via gene trap or cassette insertion. Exogenous DNA can access the cell via electroporation or chemical transformation. If the exogenous DNA has homology with chromosomal DNA it will align itself with endogenous DNA. The exogenous DNA is then inserted or disrupts the endogenous DNA via two adjacent crossing over events, known as homologous recombination. A targeting vector can use homologous recombination for insertional mutagenesis. Insertional mutagenesis of endogenous or exogenous DNA can also be carried out via DNA transposon. The DNA transposon is a mobile element that can insert itself along with additional exogenous DNA into the genome. Insertional mutagenesis of endogenous or exogenous DNA can be carried out by retroviruses. Retroviruses have a RNA viral genome that is converted into DNA by reverse transcriptase in the cytoplasm of the infected cell. Linear retroviral DNA is transported into the nucleus, and become integrated by an enzyme called integrase. Insertional mutagenesis of endogenous or exogenous DNA can also be done by retrotransposons in which an RNA intermediate is translated into double stranded DNA by reverse transcriptase, and inserting itself into the genome.

The term “transfection” means the introduction of a foreign nucleic acid into a cell. The term “transformation” means the introduction of a “foreign” (i.e. extrinsic or extracellular) gene, DNA or RNA sequence to an ES cell or pronucleus, so that the cell will express the introduced gene or sequence to produce a desired substance in a genetically modified animal. The term “infection” refers to introduction of foreign nucleic acid using a virus or viral vector.

The term “vector” is used herein to refer to a nucleic acid molecule capable of transferring or transporting another nucleic acid molecule. The transferred nucleic acid is generally linked to, for example, the vector nucleic acid molecule. A vector may include sequences that direct autonomous replication in a cell, or may include sequences sufficient to allow integration into cardiac cell DNA. Useful vectors include, for example, plasmids (e.g., DNA plasmids or RNA plasmids), transposons, cosmids, bacterial or yeast artificial chromosomes and viral vectors. Useful viral vectors include, for example, adenoviruses, retroviruses, particularly replication defective retroviruses, and lentiviruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also cardiac cell components in addition to nucleic acid(s). The term “viral vector” may also refer either to a virus or viral particle capable of transferring a nucleic acid into a cell or to the transferred nucleic acid itself. Viral vectors and transfer plasmids contain structural and/or function genetic elements that are primarily derived from a virus. The viral vector may be a hybrid vector, LTR or other nucleic acid containing both retroviral (e.g., lentiviral) sequences and non-retroviral viral sequences. A hybrid vector may refer to a vector or transfer plasmid comprising retroviral (e.g., lentiviral) sequences for reverse transcription, replication, integration and/or packaging.

The term “adenoviral vector” as used herein, refers to any adenoviral vector that includes exogenous DNA which encodes a polypeptide inserted into its genome. The vector must be capable of replicating and being packaged when any deficient essential genes are provided in trans. An adenoviral vector desirably contains at least a portion of each terminal repeat required to support the replication of the viral DNA, preferably at least about 90% of the full ITR sequence, and the DNA required to encapsidate the genome into a viral capsid. Many suitable adenoviral vectors have been described in the art. U.S. Pat. No. 6,440,944; see U.S. Pat. No. 6,040,174 (replication defective E1 deleted vectors and specialized packaging cell lines). In some embodiments, the adenoviral expression vector is one that is replication defective in normal cells. In other embodiments, an adenoviral vector refers to an adeno-associated viral (AVV) vector. In some embodiments, the adenoviral expression vector is pseudotyped to enhance targeting.

The term “retroviral vector” refers to a viral vector or plasmid containing structural and functional genetic elements, or portions thereof, that are primarily derived from a retrovirus.

The term “lentiviral vector” refers to a viral vector or plasmid containing structural and functional genetic elements, or portions thereof, that are primarily derived from a lentivirus.

The terms “lentiviral vector” or “lentiviral expression vector” may be used to refer to lentiviral transfer plasmids and/or infectious lentiviral particles. It is understood that nucleic acid sequence elements such as cloning sites, promoters, regulatory elements, heterologous nucleic acids, etc. are present in RNA form in the lentiviral particles of the invention and are present in DNA form in the DNA plasmids of the invention.

As used herein the term “equivalents thereof” refers to a polypeptide or nucleic acid sequence that differs from a reference polypeptide or nucleic acid sequence (i.e., a cyclin protein or fragment thereof consistent with embodiments of the present invention), but retains essential properties (i.e., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, deletions, additions, fusions and truncations in the polypeptide encoded by the reference sequence. Generally, differences are limited so that the sequences of the reference polypeptide and the variant are closely similar overall and, in many regions, identical.

### Immune Cells

Immune cells are part of the complex network that defends the body against pathogens and other foreign substances, including cancer cells. The cells of the immune system include, B cells, dendritic cells, granulocytes, innate lymphoid cells (ILCs), megakaryocytes, monocytes/macrophages, natural killer (NK) cells, and T cells, among others. The innate immune response, which is carried out by phagocytic cells (e.g., macrophages and cytotoxic NK cells) is the first line of defense to pathogenic exposure. Subsequently, the adaptive immune response includes antigen-specific defense mechanisms orchestrated by antigen-presenting cells (e.g., macrophages and dendritic cells). T cells (or T lymphocytes), including T regulatory cells (Tregs), T helper cells, cytotoxic T lymphocytes (CTLs), are at the core of adaptive immunity and search out and destroy foreign substances. Immune cells, for example, T cells migrate toward foreign substances in response to chemoattractant gradients provided by chemokines (e.g., CXCL12), which bind to chemokine receptors (e.g., CXCR4 and CXCR7) and provide directional cues. In some embodiments, the immune cells are T cells, NK cells, or combinations thereof. In some preferred embodiments, the immune cells are T cells.

The immune cells of the present disclosure can be isolated from any source. In some embodiments, the source of the immune cells is autologous, allogeneic, or xenographic, or combinations thereof. The immune cells may be prepared ex vivo by extracting or otherwise isolating autologous immune cells from blood, bone marrow, or other immune cell-containing organs of a patient having a cancerous tumor or other cancer, according to methods known in the art. For example, such methods include, but are not intended to be limited to apheresis techniques, specifically leukapheresis. Additionally, commercially available kits may be utilized for the extraction of T cells, such as with EASYSEP™ Human T Cell Isolation Kit available from STEMCELL™ Technologies, Inc., British Columbia, CANADA.

### Natural Killer (NK) Cells

Natural killer (NK) cells are a class of lymphocytes that typically comprise approximately 10% of the lymphocytes in a human. NK cells provide an innate cellular immune response against tumor and infected (target) cells. NK cells, which are characterized as having a CD3-/CD56+ phenotype, display a variety of activating and inhibitory cell surface receptors. NK cell inhibitory receptors predominantly engage with major histocompatibility complex class I (“MHC-I”) proteins on the surface of a normal cell to prevent NK cell activation. The MHC-I molecules define cells as “belonging” to a particular individual. It is thought that NK cells can be activated only by cells on which these “self” MHC-I molecules are missing or defective, such as is often the case for tumor or virus-infected cells.

NK cells are triggered to exert a cytotoxic effect directly against a target cell upon binding or ligation of an activating NK cell receptor to the corresponding ligand on the target cell. The cytotoxic effect is mediated by secretion of a variety of cytokines by the NK cells, which in turn stimulate and recruit other immune system agents to act against the target. Activated NK cells also lyse target cells via the secretion of the enzymes perforin and granzyme, stimulation of apoptosis-initiating receptors, and other mechanisms.

NK cells have been evaluated as an immunotherapeutic agent in the treatment of certain cancers. NK cells used for this purpose may be autologous or non-autologous (i.e., from a donor).

In one embodiment, the NK cells used in the compositions and methods herein are autologous NK cells. In one embodiment, the NK cells used in the compositions and methods herein are non-autologous NK cells.

In one embodiment, the NK cells used in the compositions and methods herein are genetically modified NK cells. NK cells can be genetically modified by insertion of genes or RNA into the cells such that the cells express one or more proteins that are not expressed by wild type NK cells. In one embodiment, the NK cells are genetically modified to express a chimeric antigen receptor (CAR). In a preferred embodiment, the CAR is specific for the cancer being targeted by the method or composition.

Non-limiting examples of modified NK cells can be found, for example, in Glienke, et al. 2015, Advantages and applications of CAR-expressing natural killer cells, Frontiers in Pharmacol. 6, article 21; PCT Patent Pub. Nos. WO 2013154760 and WO 2014055668; each of which is incorporated herein by reference in its entirety.

In some embodiments, the NK cells are an NK cell line. NK cell lines include, without limitation, NK-92, NK-YS, KHYG-1, NKL, NKG, SNK-6, and IMC-1. See, Klingemann et al. Front Immunol. 2016; 7: 91, which is incorporated herein by reference in its entirety.

“NK-29 cells” as used herein is a commercially available human cell line with the phenotypical and functional characteristics of activated natural killer cells. It is a continuously growing cell line that can be expanded to large numbers and is effective in killing tumor cells (see Gong et al., Leukemia 8(4): 652-658 (April 1994)). NK-92 cell are available from, e.g, American Tissue Culture Collection.

“NK-92 variants” as used herein are variants of NK-92 cells and include NK-92 cells modified ex vivo to express another molecule, e.g., Fc receptor such as CD16, on its surface, see e.g., U.S. Pat. No. 8,313,943, or modified to express interleukin-2 (IL-2) see e.g. U.S. Pat. No. 8,034,332.

NK-92 cells are a continuously growing cell line that can be expanded to large numbers and is effective in killing tumor cells (see Gong et al., Leukemia Vol. 8(4) PP 652-658 (April 1994) and Klingemann H-G. Development and testing of NK cell lines. In Lotze MT & Thompson AW (eds): Natural killer cells—Basic Science and Clinical applications (2010): 169-75). NK-92 cells are commercially available from, e.g., American Tissue Culture Collection. Without wishing to be bound by theory, it is contemplated that the immune system of a patient having a tumor has lost its ability to recognize tumor and/or to effectively attack and eliminate the tumor. Supplementing such a patient's immune system by the administration of immune cells that have the ability to inhibit the growth, progression and/or metastasis of a tumor should improve the patient's immune response to the tumor and enhance the patient's overall survival. In fact, the effectiveness of NK-92 cells for treating tumors, e.g., refractory or relapsed acute myeloid leukemia and Merkel cell carcinoma, and hematological malignancies is being investigated in clinical trials.

Examples of NK-92 cells are available from the American Type Culture Collection (ATCC) as ATCC CRL-2407. Examples of genetically modified NK-92 cells are available from ATCC as ATCC CRL-2408, ATCC CRL-2409, PTA-6670, PTA-6967, PTA-8837, and PTA-8836.

### T Cells

T cells are lymphocytes having T-cell receptor in the cell surface. T cells play a central role in cell-mediated immunity by tailoring the body's immune response to specific pathogens. T cells have shown promise in reducing or eliminating tumors in clinical trials. Generally, such T cells are modified and/or undergo adoptive cell transfer (ACT). ACT and variants thereof are well known in the art. See, for example, U.S. Pat. Nos. 8,383,099 and 8,034,334, which are incorporated herein by reference in their entireties.

U.S. Patent App. Pub. Nos. 2014/0065096 and 2012/0321666, incorporated herein by reference in their entireties, describe methods and compositions for T cell or NK cell treatment of cancer. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, each of which is incorporated herein by reference in its entirety.

In one embodiment, the T cells used in the compositions and methods herein are autologous T cells (i.e., derived from the patient). In one embodiment, the T cells used in the compositions and methods herein are non-autologous (heterologous; e.g. from a donor or cell line) T cells. In one embodiment, the T cell is a cell line derived from T cell(s) or cancerous/transformed T cell(s).

In a preferred embodiment, the T cell used in the methods and compositions described herein is a modified T cell. In one embodiment, the T cell is modified to express a CAR on the surface of the T cell. In a preferred embodiment, the CAR is specific for the cancer being targeted by the method or composition. In one embodiment, the T cell is modified to express a cell surface protein or cytokine. Exemplary, non-limiting examples of modified T cells are described in U.S. Pat. No. 8,906,682; PCT Patent Pub. Nos. WO 2013154760 and WO 2014055668; each of which is incorporated herein by reference in its entirety.

In one embodiment, the T cell is a T cell line. Exemplary T cell lines include T-ALL cell lines, as described in U.S. Pat. No. 5,272,082, which is incorporated herein by reference in its entirety.

### Modification

In one aspect the invention relates to an ex vivo modified immune cell overexpressing CXCR7 receptors on an outer cell surface of the modified immune cell. In another aspect the invention relates to an ex vivo modified immune cell comprising no or substantially no CXCR4 receptors on the outer cell surface of the modified immune cell. In yet another aspect the invention relates to an ex vivo modified immune cell modified to overexpress CXCR7 receptors and modified to have no or substantially no CXCR4 receptors on an cell outer surface of the modified immune cell.

One aspect of the invention relates to an ex vivo modified immune cell comprising a direct or indirect overexpression of the CXCR7 gene or gene transcript such that CXCR7 receptor expression on the outer cell surface of the cell is increased.

One aspect of the invention relates to an ex vivo modified immune cell comprising a direct or indirect suppression of the CXCR4 gene or gene transcript such that CXCR4 receptor expression on the outer cell surface of the cell is reduced or eliminated. Direct suppression refers to agents and methods which target the CXCR4 gene or CXCR4 gene transcript itself. For example, a siRNA oligonucleotide would target the CXCR4 gene transcript such that the CXCR4 gene would be expressed at normal levels, but the transcript and protein levels would be diminished such that the number of CXCR4 receptors expressed on the outer surface of the immune cell would be reduced. Another example of direct suppression the CRISPR/Cas9 system that to result in a double strand break in targeted DNA sequences such that gene expression would be reduced or eliminated. Indirect suppression may be, for example, carried out by chemical CXCR4 inhibitors such as TF14016 or cytokines such as interferon-γ (IFN-γ), IFN-α, granulocyte-macrophage colony-stimulating factor (GM-CSF), and G-CSF. Nagase et al. (2002) J. of Leukocyte Biology 71(4):711-717.

One aspect of the invention relates to an ex vivo population of modified immune cells wherein at least a portion of the modified immune cells overexpress CXCR7 on an outer cell surface of the immune cells. Another aspect of the invention relates to an ex vivo population of modified immune cells wherein at least a portion of the modified immune cells have no or substantially no CXCR4 receptors on the outer cell surface of the immune cells. Yet another aspect relates to an ex vivo population of modified immune cells wherein at least a portion of the modified immune cells overexpress CXCR7 receptors and have no or substantially no CXCR4 receptors on an cell outer surface of the modified immune cell.

It is to be understand that any method known in the art can be used to genetically modify the immune cells of the present disclosure in order to provide an immune cell overexpressing CXCR7 receptors on the cell outer cell surface, an immune cell with no or substantially no CXCR4 receptors on the cell outer cell surface, or an immune cell overexpressing CXCR7 receptors and with no or substantially no CXCR4 receptors on the cell outer surface.

In one aspect, the term “vector” intends a recombinant vector that retains the ability to infect and transduce non-dividing and/or slowly-dividing cells and integrate into the target cell's genome (e.g., immune cells). In several aspects, the vector is derived from or based on a wild-type virus or plasmid. In further aspects, the vector is derived from or based on a wild-type lentivirus. Examples of such, include without limitation, human immunodeficiency virus (HIV), equine infectious anaemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the invention need not be confined to the components of a particular virus. The viral vector may comprise components derived from two or more different viruses, and may also comprise synthetic components. In some embodiments, the vector is an episomal vector. Vector components can be manipulated to obtain desired characteristics, such as target cell specificity.

Vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,07,993; 7,419,829 and 7,442,551, incorporated herein by reference.

In one embodiment, the vector is a viral vector. In a related embodiment, the viral vector is selected from the group consisting of a lentiviral vector, retroviral vector, adenovirus vector, adeno-associated virus vector, episomal vector, and alphavirus vector. In yet a further embodiment, the viral vector is a lentiviral vector.

Non-viral vectors may include a plasmid that comprises a heterologous polynucleotide capable of being delivered to a target cell, either in vitro, in vivo or ex-vivo. The heterologous polynucleotide can comprise a sequence of interest (e.g., CXCR7) and can be operably linked to one or more regulatory elements and may control the transcription of the nucleic acid sequence of interest (e.g., CXCR7).

It is to be understand that any method known in the art can be used to genetically modify the immune cells of the present disclosure in order to provide an immune cell with no or substantially no CXCR4 receptors on the cell outer cell surface. Gene knockdown refers to the temporary decrease in gene expression in a cell. One commonly used method for gene knockdown is RNAi (e.g., short interfering RNA (siRNA) and short hairpin RNA (shRNA)), which typically does not completely shut off the genes, but reduces transcript and protein levels. Ketting (2011) Dev. Cell 20(2): 148-161. Using these RNAi methods, gene function is reduced, but not eliminated. On the other hand, gene editing (e.g., genetic engineering in which DNA is inserted, replaced, or removed from the genome) can be used to make targeted, permanent changes to genes such that gene function is completely or substantially eliminated (“knockout”). Commonly used methods for knockout include, but are not limited to, Transcription Activator-Like Effector Nucleases (TALENs) and Clustered, Regularly Interspaced Palindromic Repeat Associated (CRISPR-Cas) proteins and Zinc Finger Nucleases (ZFN). Bogdanove & Voytas (2011) Science 333(6051): 1843-1846; Shalem, et al. (2014) Science 343:84-87; and U.S. Pat. No. 8,697,359. Urnov et al. (2010) Nature Reviews 11:636-646.

RNA interference “RNAi” is mediated by double stranded RNA (dsRNA) molecules that have sequence-specific homology to their “target” nucleic acid sequences. Caplen, N. J., et al., (2001) Proc. Natl. Acad. Sci. USA 98:9742-9747. In certain embodiments of the present invention, the mediators of RNA-dependent CXCR4 silencing are 21-25 nucleotide “small interfering” RNA duplexes (siRNAs). The siRNAs are derived from the processing of dsRNA by an RNase enzyme known as Dicer. Bernstein, E., et al., (2001) Nature 409:363-366. siRNA duplex products are recruited into a multi-protein siRNA complex termed RISC (RNA Induced Silencing Complex). RISC is then believed to be guided to a target nucleic acid (suitably mRNA), where the siRNA duplex interacts in a sequence-specific way to mediate cleavage in a catalytic fashion. Bernstein, E., et al., (2001) Nature 409:363-366; Boutla, A., et al., (2001) Curr. Biol. 11:1776-1780 (2001). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

In some embodiments, a method for modulating CXCR4 receptor levels on the immune cells comprises an aptamer-interference RNA (RNAi) molecule wherein said molecule is targeted to CXCR4. In another embodiment, the interference RNA comprises at least one of a short interfering RNA (siRNA); a micro interfering RNA (miRNA); a small temporal RNA (stRNA); or a short hairpin RNA (shRNA). In a some embodiments, the RNAi is a siRNA or a shRNA.

Engineered nucleases, including CRISPR/Cas nuclease systems, zinc finger nucleases (ZFNs), TALENs and homing endonucleases designed to specifically bind to target DNA sites are also useful in genome engineering. For example, zinc finger nucleases (ZFNs) are proteins comprising engineered site-specific zinc fingers fused to a nuclease domain. Such ZFNs and TALENs have been successfully used for genome modification in a variety of different species. See, for example, U.S. Pat. Publications 2003/0232410; 2005/0208489; 2005/0026157; 2005/0064474; 2006/0188987; 2006/0063231; 2011/0301073; 2013/0177983; 2013/0177960; and International Publication WO 07/014275, the disclosures of which are incorporated by reference in their entireties for all purposes. These engineered nucleases can create a double-strand break (DSB) at a specified nucleotide sequence which increases the frequency of homologous recombination at the targeted locus by more than 1000-fold. Thus, engineered nucleases can be used to exploit the homology-directed repair (HDR) system and facilitate targeted integration of transgenes into the genome of cells. In addition, the inaccurate repair of a site-specific DSB by non-homologous end joining (NHEJ) can also result in gene disruption. It is contemplated that CRISPR/Cas, TALEN, or ZFN can be used to insert CXCR7 gene into an immune cells (e.g., T-cells) to act, at least in part, as a decoy to bind and degrade the CXCL12-induced fugetactic wall in order to allow immune cells to detect and destroy tumor cells. It is also contemplated, that CRISPR/Cas, TALEN, or ZFN can be used to activate endogenous CXCR7. It is further contemplated that CRISPR/Cas, TALEN, or ZFN nuclease and/or targeting of CXCR4 in immune cells(e.g., T-cells) can be used to effectively evade the fugetactic wall created by tumors overexpressing CXCL12 and allow the immune cells to reach and kill the tumor cells, thereby treating cancer.

In some embodiments, a CRISPR/Cas system is used to introduce into an immune cell DNA molecules encoding one or more gene products (i.e., CXCR7), wherein the CRISPR/Cas system comprises a CRIPSR/Cas nuclease and an engineered crRNA/tracrRNA (or single guide RNA) are employed. See, U.S. Pat. No. 8,697,359. In other embodiments, a CRISPR/Cas system that binds to target site in a region of interest in a CXCR4 gene in a genome, wherein the CRISPR/Cas system comprises a CRIPSR/Cas nuclease and an engineered crRNA/tracrRNA (or single guide RNA) are employed. See, U.S. Pat. Publications 2015/0056705.

In another aspect, a polynucleotide encoding a nuclease is provided, for example a polynucleotides encoding one or more zinc finger nucleases (ZFNs), one or more TALENs, one or more meganucleases and/or one or more CRISPR/Case nucleases. The polynucleotide can comprise DNA, RNA or combinations thereof. In certain embodiments, the polynucleotide comprises a plasmid. In other embodiments, the polynucleotide encoding the nuclease comprises mRNA.

In some embodiments, the modified immune cells have increased amounts of CXCR7 on the outer cell surface, for example, the immune cell has 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 100% or more, 200% or more, 300% or more (and any sub value or sub range between 10% and 500%) CXCR7 on the outer cell surface as compared to average number of CXCR7 receptors on an unmodified immune cell.

In some embodiments, the modified immune cells have no CXCR4 on the outer cell surface. In other embodiments, the modified immune cells have substantially no CXCR4 on the outer cell surface, for example, the immune cell has 50% or less, 45% or less, 40% or less, 35% or less, 30% or less, 25% or less, 20% or less, 15% or less, 10% or less, 5% or less, or 1% or less (and any sub value or subrange between 50% and 1%) of the amount of CXCR4 receptors on the outer cell surface as compared to average number of CXCR4 receptors on an unmodified immune cell.

The number or average number of receptors expressed by a cell or cell population can be determined by any method known in the art. By way of non-limiting example, these include fluorescence-activated cell sorting (FACS), Western blotting, reverse transcriptase polymerase chain reaction (RT-PCR), real time RT-PCR, visual analysis (e.g., cell staining), and the like.

Genes may be delivered to the cell by a variety of mechanisms commonly known to those of skill in the art. Viral constructs can be delivered through the production of a virus in a suitable host cell. Virus is then harvested from the host cell and contacted with the target cell. Viral and non-viral vectors capable of expressing genes of interest can be delivered to a targeted cell via DNA/liposome complexes, micelles and targeted viral protein-DNA complexes. Liposomes that also comprise a targeting antibody or fragment thereof can be used in the methods of this invention. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

Other methods of delivering vectors encoding genes of the current invention include but are not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals (e.g., mammals). In vitro expression of a protein, fusion, polypeptide fragment, or mutant encoded by cloned DNA may also be used. Those skilled in the art of molecular biology will understand that a wide variety of expression systems and purification systems may be used to produce recombinant proteins and fragments thereof.

### CXCR7

As discussed, an immune cells (e.g., T cells) may be modified to increase expression of CXCR7. CXCR7 is a chemokine receptor for CXCL12 and CXCL11 that is thought to act, at least in part, as a “decoy” receptor. Singh et al. (2013) Cytokine Growth Factor Rev. 24(1):41-49. CXCR7 is also known as Atypical Chemokine Receptor 3 (ACK3).

Amino acid sequences for CXCR7 and nucleotide sequences encoding CXCR7 polypeptides, from a variety of species, are known in the art. See, e.g.: (1) GenBank Accession No. NP_064707.1 (Homo sapiens 362 amino acid atypical chemokine receptor 3); (2) GenBank Accession No. NP_001258536.1 (Mus musculus 362 amino acid atypical chemokine receptor 3); (3) GenBank Accession No. NM_020311.2 (nucleotide sequence encoding the Homo sapiens atypical chemokine receptor 3 (ACKR3)); (4) GenBank Accession No. NM_007722.4 (nucleotide sequence encoding the Mus musculus atypical chemokine receptor 3, transcript variant 2).

In some embodiments, a suitable CXCR7 nucleic acid comprises a nucleotide sequence encoding a CXCR7 polypeptide, wherein the suitable nucleotide sequence comprises an nucleotide sequence having at least about 80%, at least about 85%, at least about 90%, at least about 95%, at least about 99%, or 100% nucleotide sequence identity of the sequences disclosed herein (or any sub value or sub range there between).

In some embodiments, a suitable CXCR7 polypeptide comprises an amino sequence encoding a CXCR7 polypeptide, wherein the suitable amino acid sequence comprises an polypeptide sequence having at least about 80%, at least about 85%, at least about 90%, at least about 95%, at least about 99%, or 100% amino acid sequence identity of the sequences disclosed herein (or any sub value or sub range there between).

### CXCR4

As discussed, an immune cells (e.g., T cells) may be modified have reduced or no expression of CXCR4. CXCR4 is a chemokine receptor for CXCL12 and upon binding of CXCL12 to CXCR4 induces intracellular signaling related to chemotaxis, cell survival and/or proliferation, among others. Teicher et al. (2010) Clin. Cancer Res. 16:2927-2931.

Amino acid sequences for CXCR4 and nucleotide sequences encoding CXCR4 polypeptides, from a variety of species, are known in the art. See, e.g.: (1) GenBank Accession No. CAA12166.1 (Homo sapiens 360 amino acid CXCR4); (2) GenBank Accession No. NP_034041.2 (Mus musculus 359 amino acid Cxcr4); (3) GenBank Accession No. NM_003467.2 (nucleotide sequence encoding the Homo sapiens chemokine receptor 4 (CXCR4), transcript variant 2); (4) GenBank Accession No. NM_001008540.1 (nucleotide sequence encoding the Homo sapiens chemokine receptor 4 (CXCR4), transcript variant 1); (5) GenBank Accession No. NM_009911.3 (nucleotide sequence encoding the Mus musculus chemokine receptor 4 (Cxcr4)). The sequence and structure of the human CXCR4 is known; see e.g., GenBank Accession Nos. NM_003467 and NM 001008540 for the nucleotide sequence and NP_003458 The nucleotide and polypeptide sequences of human SDF-1α are set forth in GenBank Accession Nos. NM.sub.-000609 and NP.sub.-000600, respectively.

In some embodiments, a suitable CXCR4 nucleic acid comprises a nucleotide sequence encoding a CXCR4 polypeptide, wherein the suitable nucleotide sequence comprises a nucleotide sequence having at least about 80%, at least about 85%, at least about 90%, at least about 95%, at least about 99%, or 100% nucleotide sequence identity of the sequences disclosed herein (or any sub value or sub range there between).

In some embodiments, a suitable CXCR4 polypeptide comprises an amino sequence encoding a CXCR4 polypeptide, wherein the suitable amino acid sequence comprises a polypeptide sequence having at least about 80%, at least about 85%, at least about 90%, at least about 95%, at least about 99%, or 100% amino acid sequence identity of the sequences disclosed herein (or any sub value or sub range there between).

In some embodiments, the modified immune cells have no CXCR4 on the outer cell surface. In other embodiments, the modified immune cells have substantially no CXCR4 on the outer cell surface, for example, the immune cell has 50% or less, 45% or less, 40% or less, 35% or less, 30% or less, 25% or less, 20% or less, 15% or less, 10% or less, 5% or less, or 1% or less of the amount of CXCR4 receptors on the outer cell surface as compared to average number of CXCR4 receptors on an unmodified immune cell (or any sub value or sub range there between).

### Homing Receptor

In some embodiments, the immune cells are modified to express a tumor cell homing receptor on the outer cell surface of the immune cell. The homing receptor may be, for example, a chimeric antigen receptor, an Fc receptor, or combinations thereof. In some embodiments the CAR targets a cancer-associated antigen. In other embodiments, at least a portion of the immune cells express an endogenous tumor cell homing receptor that is not CXCR4.

In one aspect of the disclosure, the immune cell is modified to express a chimeric antigen receptor (CAR). In some embodiments, the immune cell is transformed with a nucleic acid encoding a CAR, wherein the CAR is expressed on the outer cell surface of the immune cell. In some embodiments, the immune cell is a T cell, for example, an activated T cell.

In some embodiments, the immune cells are modified to express the tumor homing receptor prior to being modified to express no or substantially no CXCR4 on the cell surface.

In some embodiments, the immune cells are modified to express the tumor homing receptor after being modified to express no or substantially no CXCR4 on the cell surface. In some embodiments, the immune cells are modified to express the tumor homing receptor prior and to express no or substantially no CXCR4 on the cell surface at the same time or substantially the same time.

In some embodiments, the immune cells are transformed with a nucleic acid encoding a CAR, and express the CAR on the outer cell surface. In some embodiments, the immune cell is a T cell, for example, an activated T cell.

Any CAR known to one of skill in the art now or in the future is encompassed by the present disclosure. In one embodiment, the CAR is specific for a tumor-specific antigen. Tumor-specific antigens can also be referred to as cancer-specific antigen. In one embodiment, the CAR is specific for a tumor-associated antigen. Tumor-associated antigens can also be referred to as cancer-associated antigen. A tumor-specific antigen is a protein or other molecule that is unique to cancer cells, while a tumor-associated antigen is an antigen that is highly correlated with certain tumor cells and typically are found at higher levels on a tumor cell as compared to on a normal cell. Tumor-specific antigens are described, by way of non-limiting example, in U.S. Pat. No. 8,399,645, U.S. Pat. No. 7,098,008; WO 1999/024566; WO 2000/020460; and WO 2011/163401, each of which is incorporated herein by reference in its entirety. In addition, non-limiting examples of some known CARs are provided in Table 2. In one embodiment, the CAR targets a tumor-associated antigen selected from the group consisting of α-folate receptor, CAIX, CD19, CD20, CD30, CD33, CEA, EGP-2, erb-B2, erb-B 2,3,4, FBP, GD2, GD3, Her2/neu, IL-13R-a2, k-light chain, LeY, MAGE-AL Mesothelin, and PSMA.

In some embodiments, the CAR recognizes an antigen associated with a specific cancer type selected from the group consisting of ovarian cancer, renal cell carcinoma, B-cell malignancies, Acute lymphoblastic leukemia (ALL), chronic lymphocytic leukemia (CLL), B-cell malignancies, refractory follicular lymphoma, mantle cell lymphoma, indolent B cell lyphoma, acute myeloid leukemia (AML), Hodgkin lymphoma, cervical carcinoma, breast cancer, colorectal cancer, prostate cancer, neuroblastoma, melanoma, rhabdomyosarcoma, medulloblastoma, adenocarcinomas, and tumor neovasculature.

TABLE 2
Examples of Chimeric Antigen Receptors

| | | | CARs |
|---|---|---|---|
| Target antigen | Associated malignancy | Receptor type | generation |
| α-Folate receptor | Ovarian cancer | ScFv-FcεRIγCAIX | First |
| CAIX | Renal cell carcinoma | ScFv-FcεRIγ | First |
| CAIX | Renal cell carcinoma | ScFv-FcεRIγ | Second |
| CD19 | B-cell malignancies | ScFv-CD3ζ (EBV) | First |
| CD19 | B-cell malignancies, CLL | ScFv-CD3ζ | First |
| CD19 | B-ALL | ScFv-CD28-CD3ζ | Second |
| CD19 | ALL | CD3ζ(EBV) | First |
| CD19 | ALL post-HSCT | ScFv-CD28-CD3ζ | Second |
| CD19 | Leukemia, lymphoma, CLL | ScFv-CD28-CD3ζ vs. | First and |
| | | CD3ζ | Second |
| CD19 | B-cell malignancies | ScFv-CD28-CD3ζ | Second |
| CD19 | B-cell malignancies post- | ScFv-CD28-CD3ζ | Second |
| | HSCT | | |
| CD19 | Refractory Follicular | ScFv-CD3ζ | First |
| | Lymphoma | | |
| CD19 | B-NHL | ScFv-CD3ζ | First |
| CD19 | B-lineage lymphoid | ScFv-CD28-CD3ζ | Second |
| | malignancies post-UCBT | | |
| CD19 | CLL, B-NHL | ScFv-CD28-CD3ζ | Second |
| CD19 | B-cell malignancies, CLL, B- | ScFv-CD28-CD3ζ | Second |
| | NHL | | |
| CD19 | ALL, lymphoma | ScFv-41BB-CD3ζ vs | First and |
| | | CD3ζ | Second |
| CD19 | ALL | ScFv-41BB-CD3ζ | Second |
| CD19 | B-cell malignancies | ScFv-CD3ζ (Influenza | First |
| | | MP-1) | |
| CD19 | B-cell malignancies | ScFv-CD3ζ (VZV) | First |
| CD20 | Lymphomas | ScFv-CD28-CD3ζ | Second |
| CD20 | B-cell malignancies | ScFv-CD4-CD3ζ | Second |
| CD20 | B-cell lymphomas | ScFv-CD3ζ | First |
| CD20 | Mantle cell lymphoma | ScFv-CD3ζ | First |
| CD20 | Mantle cell lymphoma, | CD3 ζ/CD137/CD28 | Third |
| | indolent B-NHL | | |
| CD20 | indolent B cell lymphomas | ScFv-CD28-CD3ζ | Second |
| CD20 | Indolent B cell lymphomas | ScFv-CD28-41BB- | Third |
| | | CD3ζ | |
| CD22 | B-cell malignancies | ScFV-CD4-CD3ζ | Second |
| CD30 | Lymphomas | ScFv-FcεRIγ | First |
| CD30 | Hodgkin lymphoma | ScFv-CD3ζ (EBV) | First |
| CD33 | AML | ScFv-CD28-CD3ζ | Second |
| CD33 | AML | ScFv-41BB-CD3ζ | Second |
| CD44v7/8 | Cervical carcinoma | ScFv-CD8-CD3ζ | Second |
| CEA | Breast cancer | ScFv-CD28-CD3ζ | Second |
| CEA | Colorectal cancer | ScFv-CD3ζ | First |
| CEA | Colorectal cancer | ScFv-FceRIγ | First |
| CEA | Colorectal cancer | ScFv-CD3ζ | First |
| CEA | Colorectal cancer | ScFv-CD28-CD3ζ | Second |
| CEA | Colorectal cancer | ScFv-CD28-CD3ζ | Second |
| EGP-2 | Multiple malignancies | scFv-CD3ζ | First |
| EGP-2 | Multiple malignancies | scFv-FcεRIγ | First |
| EGP-40 | Colorectal cancer | scFv-FcεRIγ | First |
| erb-B2 | Colorectal cancer | CD28/4-1BB-CD3ζ | Third |
| erb-B2 | Breast and others | ScFv-CD28-CD3ζ | Second |
| erb-B2 | Breast and others | ScFv-CD28-CD3ζ | Second |
| | | (Influenza) | |
| erb-B2 | Breast and others | ScFv-CD28mut-CD3ζ | Second |
| erb-B2 | Prostate cancer | ScFv-FcεRIγ | First |
| erb-B 2,3,4 | Breast and others | Heregulin-CD3ζ | Second |
| erb-B 2,3,4 | Breast and others | ScFv-CD3ζ | First |
| FBP | Ovarian cancer | ScFv-FcεRIγ | First |
| FBP | Ovarian cancer | ScFv-FcεRIγ | First |
| | | (alloantigen) | |
| Fetal | Rhabdomyosarcoma | ScFv-CD3ζ | First |
| acetylcholine | | | |
| receptor | | | |
| GD2 | Neuroblastoma | ScFv-CD28 | First |
| GD2 | Neuroblastoma | ScFv-CD3ζ | First |
| GD2 | Neuroblastoma | ScFv-CD3ζ | First |
| GD2 | Neuroblastoma | ScFv-CD28-OX40- | Third |
| | | CD3ζ | |
| GD2 | Neuroblastoma | ScFv-CD3ζ (VZV) | First |
| GD3 | Melanoma | ScFv-CD3ζ | First |
| GD3 | Melanoma | ScFv-CD3ζ | First |
| Her2/neu | Medulloblastoma | ScFv-CD3ζ | First |
| Her2/neu | Lung malignancy | ScFv-CD28-CD3ζ | Second |
| Her2/neu | Advanced osteosarcoma | ScFv-CD28-CD3ζ | Second |
| Her2/neu | Glioblastoma | ScFv-CD28-CD3ζ | Second |
| IL-13R-a2 | Glioma | IL-13-CD28-4-1BB- | Third |
| | | CD3ζ | |
| IL-13R-a2 | Glioblastoma | IL-13-CD3ζ | Second |
| IL-13R-a2 | Medulloblastoma | IL-13-CD3ζ | Second |
| KDR | Tumor neovasculature | ScFv-FcεRIγ | First |
| k-light chain | B-cell malignancies | ScFv-CD3ζ | First |
| k-light chain | (B-NHL, CLL) | ScFv-CD28-CD3ζ vs | Second |
| | | CD3ζ | |
| LeY | Carcinomas | ScFv-FcεRIγ | First |
| LeY | Epithelial derived tumors | ScFv-CD28-CD3ζ | Second |
| L1 cell adhesion | Neuroblastoma | ScFv-CD3ζ | First |
| molecule | | | |
| MAGE-A1 | Melanoma | ScFV-CD4-FcεRIγ | Second |
| MAGE-A1 | Melanoma | ScFV-CD28-FcεRIγ | Second |
| Mesothelin | Various tumors | ScFv-CD28-CD3ζ | Second |
| Mesothelin | Various tumors | ScFv-41BB-CD3ζ | Second |
| Mesothelin | Various tumors | ScFv-CD28-41BB- | Third |
| | | CD3ζ | |
| Murine CMV | Murine CMV | Ly49H-CD3ζ | Second |
| infected cells | | | |
| MUC1 | Breast, Ovary | ScFV-CD28-OX40- | Third |
| | | CD3ζ | |
| NKG2D ligands | Various tumors | NKG2D-CD3ζ | First |
| Oncofetal antigen | Various tumors | ScFV-CD3ζ | First |
| (h5T4) | | (vaccination) | |
| PSCA | Prostate carcinoma | ScFv-b2c-CD3ζ | Second |
| PSMA | Prostate/tumor vasculature | ScFv-CD3ζ | First |
| PSMA | Prostate/tumor vasculature | ScFv-CD28-CD3ζ | Second |
| PSMA | Prostate/tumor vasculature | ScFv-CD3ζ | First |
| TAA targeted by | Various tumors | FceRI-CD28-CD3ζ (+ | Third |
| mAb IgE | | a-TAA IgE mAb) | |
| TAG-72 | Adenocarcinomas | scFv-CD3ζ | First |
| VEGF-R2 | Tumor neovasculature | scFv-CD3ζ | First |

### Anti-Fugetactic Agents

Many tumors have fugetactic effects, e.g. on immune cells, due to chemokines secreted by the tumor cells. High concentrations of the chemokines secreted by the tumor cells can have fugetactic (chemorepellant) effects on cells, whereas lower concentrations do not have such effects or even result in chemoattraction. For example, T-cells are repelled by CXCL12 (SDF-1) by a concentration-dependent and CXCR4 receptor-mediated mechanism. This invention is predicated on the surprising discovery that anti-fugetactic agents as described herein reduce the fugetactic effects of the tumors, thereby allowing immune cells and other anti-cancer agents to better access and kill the tumor cells.

The anti-fugetactic agent may be any such agent known in the art, for example an anti-fugetactic agent as described in U.S. Patent Application Publication No. 2008/0300165, which is hereby incorporated by reference in its entirety.

Anti-fugetactic agents include any agents that specifically inhibit chemokine and/or chemokine receptor dimerization, thereby blocking the chemorepellent response to a fugetactic agent. Certain chemokines, including IL-8 and CXCL12 can also serve as chemorepellents at high concentrations (e.g., above 100 nM) where much of the chemokine exists as a dimer. Dimerization of the chemokine elicits a differential response in cells, causing dimerization of chemokine receptors, an activity which is interpreted as a chemorepellent signal. Blocking the chemorepellent effect of high concentrations of a chemokine secreted by a tumor can be accomplished, for example, by anti-fugetactic agents which inhibit chemokine dimer formation or chemokine receptor dimer formation. For example, antibodies that target and block chemokine receptor dimerization, for example, by interfering with the dimerization domains or ligand binding can be anti-fugetactic agents. Anti-fugetactic agents that act via other mechanisms of action, e.g. that reduce the amount of fugetactic cytokine secreted by the cells, inhibit dimerization, and/or inhibit binding of the chemokine to a target receptor, are also encompassed by the present invention. Where desired, this effect can be achieved without inhibiting the chemotactic action of monomeric chemokine.

In some embodiments, the anti-fugetactic agent further may be any such agent known in the art, for example, an anti-fugetactic agent as described in U.S. Patent Application Publication No. 2008/0300165, which is hereby incorporated by reference in its entirety.

In other embodiments, the anti-fugetactic agent is a CXCR4 antagonist, CXCR3 antagonist, CXCR4/CXCL12 antagonist or selective PKC inhibitor.

The CXCR4 antagonist can be but is not limited to AMD3100, KRH-1636, T-20, T-22, T-140, TE-14011, T-14012, or TN14003, or an antibody that interferes with the dimerization of CXCR4. Additional CXCR4 antagonists are described, for example, in U.S. Patent Pub. No. 2014/0219952 and Debnath et al. Theranostics, 2013; 3(1): 47-75, each of which is incorporated herein by reference in its entirety, and include TG-0054 (burixafor), AMD3465, NIBR1816, AMD070, and derivatives thereof.

The CXCR3 antagonist can be but is not limited to TAK-779, AK602, or SCH-351125, or an antibody that interferes with the dimerization of CXCR3.

The CXCR4/CXCL12 antagonist can be but is not limited to Tannic acid, NSC 651016, or an antibody that interferes with the dimerization of CXCR4 and/or CXCL12.

The selective PKC inhibitor can be but is not limited to thalidomide or GF 109230X.

In a preferred embodiment, the anti-fugetactic agent is AMD3100 (plerixafor). AMD3100 is described in U.S. Pat. No. 5,583,131, which is incorporated by reference herein in its entirety.

In one embodiment, the anti-fugetactic agent is an AMD3100 derivative. AMD3100 derivatives include, but are not limited to, those found in U.S. Pat. Nos. 7,935,692 and 5,583,131 (USRE42152), each of which is incorporated herein by reference in its entirety.

In one embodiment, the anti-fugetactic agent is coupled with a molecule that allows targeting of a tumor. In one embodiment, the anti-fugetactic agent is coupled with (e.g., bound to) an antibody specific for the tumor to be targeted. In one embodiment, the anti-fugetactic agent coupled to the molecule that allows targeting of the tumor is administered systemically.

CXCL12 expression by a tumor may also promote tumor growth, angiogenesis, and metastasis. Accordingly, methods for inhibiting tumor growth, angiogenesis, and metastasis are contemplated by this invention.

In one embodiment, the anti-fugetactic agent is administered in combination with an additional compound that enhances the anti-fugetactic activity of the agent. In one embodiment, the additional compound is granulocyte colony stimulating factor (G-CSF). In one embodiment, G-CSF is not administered.

### Anti-Cancer Agents

In one aspect of the present invention, the modified immune cells are administered in combination with at least one additional anti-cancer agent. In one embodiment, the at least one additional anti-cancer agent is a chemotherapy agent. In one embodiment, the at least one additional anti-cancer agent is a radiotherapy agent. In one embodiment, the at least one additional anti-cancer agent is an immunotherapy agent. In one embodiment, the at least one additional anti-cancer agent is a combination of two or more of the above.

### Chemotherapy Agents

In one aspect of the present invention, the modified immune cells are administered in combination with a chemotherapy agent. The chemotherapy agent may be any agent having a therapeutic effect on one or more types of cancer. Many chemotherapy agents are currently known in the art. Types of chemotherapy drugs include, by way of non-limiting example, alkylating agents, antimetabolites, anti-tumor antibiotics, totpoisomerase inhibitors, mitotic inhibitors, corticosteroids, and the like.

Non-limiting examples of chemotherapy drugs include: nitrogen mustards, such as mechlorethamine (nitrogen mustard), chlorambucil, cyclophosphamide (Cytoxan®), ifosfamide, and melphalan); Nitrosoureas, such as streptozocin, carmustine (BCNU), and lomustine; alkyl sulfonates, such as busulfan; Triazines, such as dacarbazine (DTIC) and temozolomide (Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); Capecitabine (Xeloda®); Cytarabine (Ara-C®); Floxuridine; Fludarabine; Gemcitabine (Gemzar®); Hydroxyurea; Methotrexate; Pemetrexed (Alimta®); anthracyclines, such as Daunorubicin, Doxorubicin (Adriamycin®), Epirubicin, Idarubicin; Actinomycin-D; Bleomycin; Mitomycin-C; Mitoxantrone; Topotecan; Irinotecan (CPT-11); Etoposide (VP-16); Teniposide; Mitoxantrone; Taxanes: paclitaxel (Taxol®) and docetaxel (Taxotere®); Epothilones: ixabepilone (Ixempra®); Vinca alkaloids: vinblastine (Velban®), vincristine (Oncovin®), and vinorelbine (Navelbine®); Estramustine (Emcyt®); Prednisone; Methylprednisolone (Solumedrol®); Dexamethasone (Decadron®); L-asparaginase; bortezomib (Velcade®). Additional chemotherapy agents are listed, for example, in U.S. Patent Application Pub. No. 2008/0300165, which is incorporated herein by reference in its entirety.

Doses and administration protocols for chemotherapy drugs are well-known in the art. The skilled clinician can readily determine the proper dosing regimen to be used, based on factors including the chemotherapy agent(s) administered, type of cancer being treated, stage of the cancer, age and condition of the patient, patient size, location of the tumor, and the like.

### Radiotherapy Agents

In one aspect of the present invention, the modified immune cells administered in combination with a radiotherapeutic agent. The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In one embodiment, the radiotherapeutic agent is delivered by a machine outside of the body (external-beam radiation therapy). In a preferred embodiment, the radiotherapeutic agent is placed in the body near the tumor/cancer cells (brachytherapy) or is a systemic radiation therapy.

External-beam radiation therapy may be administered by any means. Exemplary, non-limiting types of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent. Exemplary, non-limiting types of internal radiation therapy include any radioactive agents that can be placed proximal to or within the tumor, such as Radium-226 (Ra-226), Cobalt-60 (Co-60), Cesium-137 (Cs-137), cesium-131, Iridium-192 (Ir-192), Gold-198 (Au-198), Iodine-125 (1-125), palladium-103, yttrium-90, etc. Such agents may be administered by seeds, needles, or any other route of administration, and my be temporary or permanent.

Systemic radiation therapy may be by any technique or agent. Exemplary, non-limiting types of systemic radiation therapy include radioactive iodine, ibritumomab tiuxetan (Zevalin®), tositumomab and iodine I 131 tositumomab (Bexxar®), samarium-153-lexidronam (Quadramet®), strontium-89 chloride (Metastron®), metaiodobenzylguanidine, lutetium-177, yttrium-90, strontium-89, and the like.

In one embodiment, a radiosensitizing agent is also administered to the patient. Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like.

### Immunotherapy Agents

### Anti-Cancer Vaccines

In one aspect of the present invention, the modified immune cells are administered in combination with an anti-cancer vaccine (also called cancer vaccine). Anti-cancer vaccines are vaccines that either treat existing cancer or prevent development of a cancer by stimulating an immune reaction to kill the cancer cells. In a preferred embodiment, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gp100 melanoma vaccine, and any other vaccine that stimulates an immune response to cancer cells when administered to a patient.

### Antibodies

Immunotherapy also refers to treatment with anti-tumor antibodies. That is, antibodies specific for a particular type of cancer (e.g., a cell surface protein expressed by the target cancer cells) can be administered to a patient having cancer. The antibodies may be monoclonal antibodies, polyclonal antibodies, chimeric antibodies, antibody fragments, human antibodies, humanized antibodies, or non-human antibodies (e.g. murine, goat, primate, etc.). The therapeutic antibody may be specific for any tumor-specific or tumor-associated antigen. See, e.g. Scott et al., Cancer Immunity 2012, 12:14, which is incorporated herein by reference in its entirety.

In one embodiment, the immunotherapy agent is an anti-cancer antibody. Non-limiting examples include trastuzumab (Herceptin®), bevacizumab (Avastin®), cetuximab (Erbitux®), panitumumab (Vectibix®), ipilimumab (Yervoy®), rituximab (Rituxan®), alemtuzumab (Campath®), ofatumumab (Arzerra®), gemtuzumab ozogamicin (Mylotarg®), brentuximab vedotin (Adcetris®), ⁹⁰Y-ibritumomab tiuxetan (Zevalin®), and ¹³¹I-tositumomab (Bexxar®).

Additional, non-limiting antibodies are provided in Table 1.

TABLE 1
Anti-cancer antibodies

| Proprietary | | | Indication first approved or |
|---|---|---|---|
| name | Trade name | Target; Format | reviewed |
| Necitumumab | (Pending) | EGFR; Human IgG1 | Non-small cell lung cancer |
| Nivolumab | Opdivo | PD1; Human IgG4 | Melanoma |
| Dinutilximab | (Pending) | GD2; Chimeric | Neuroblastoma |
| | | IgG1 | |
| Blinatumomab | Blincyto | CD19, CD3; Murine | Acute lymphoblastic leukemia |
| | | bispecific tandem | |
| | | scFv | |
| Pembrolizumab | Keytruda | PD1; Humanized | Melanoma |
| | | IgG4 | |
| Ramucirumab | Cyramza | VEGFR2; Human | Gastric cancer |
| | | IgG1 | |
| Obinutuzumab | Gazyva | CD20; Humanized | Chronic lymphocytic |
| | | IgG1; | leukemia |
| | | Glycoengineered | |
| Ado-trastuzumab | Kadcy1a | HER2; humanized | Breast cancer |
| emtansine | | IgG1; | |
| | | immunoconjugate | |
| Pertuzumab | Perjeta | HER2; humanized | Breast Cancer |
| | | IgG1 | |
| Brentuximab | Adcetris | CD30; Chimeric | Hodgkin lymphoma, systemic |
| vedotin | | IgG1; | anaplastic large cell |
| | | immunoconjugate | lymphoma |
| Ipilimumab | Yervoy | CTLA-4; Human | Metastatic melanoma |
| | | IgG1 | |
| Ofatumumab | Arzerra | CD20; Human IgG1 | Chronic lymphocytic |
| | | | leukemia |

### Immune Checkpoint Inhibitors

In one embodiment, the immunotherapy agent is a checkpoint inhibitor. Immune checkpoint proteins are made by some types of immune system cells, such as T cells, and some cancer cells. These proteins, which can prevent T cells from killing cancer cells, are targeted by checkpoint inhibitors. Checkpoint inhibitors increase the T cells' ability to kill the cancer cells. Examples of checkpoint proteins found on T cells or cancer cells include PD-1/PD-L1 and CTLA-4/B7-1/B7-2.

In one embodiment, the checkpoint inhibitor is an antibody to a checkpoint protein, e.g., PD-1, PDL-1, or CTLA-4. Checkpoint inhibitor antibodies include, without limitation, BMS-936559, MPDL3280A, MedI-4736, Lambrolizumab, Alemtuzumab, Atezolizumab, Ipilimumab, Nivolumab, Ofatumumab, Pembrolizumab, and Rituximab.

### Cytokines

In one embodiment, the immunotherapy agent is a cytokine. Cytokines stimulate the patient's immune response. Cytokines include interferons and interleukins. In one embodiment, the cytokine is interleukin-2. In one embodiment, the cytokine is interferon-alpha.

### Cancers

Cancers or tumors that can be treated by the cells, compositions and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In important embodiments, cancers or tumors escaping immune recognition include glioma, colon carcinoma, colorectal cancer, lymphoid cell-derived leukemia, choriocarcinoma, and melanoma. In one embodiment, the cancer is breast cancer, preferably inflammatory breast cancer.

In a preferred embodiment, the tumor is a solid tumor. In one embodiment, the tumor is a leukemia. In an especially preferred embodiment, the tumor over-expresses CXCL12. In one embodiment, tumor expression of CXCL12 can be evaluated prior to administration of a composition as described herein. For example, a patient having a tumor that is determined to express or over-express CXCL12 will be treated using a method and/or composition as described herein.

In one embodiment, the tumor is a brain tumor. It is contemplated that a brain tumor, e.g., an inoperable brain tumor, can be injected with a composition described herein. In one embodiment, the modified immune cells are administered directly to a brain tumor via a catheter into a blood vessel within or proximal to the brain tumor. Further discussion of catheter or microcatheter administration is described below.

In one embodiment, the cancer is inflammatory breast cancer. Inflammatory breast cancer is a rare and very aggressive disease in which cancer cells block lymph vessels in the skin of the breast. This type of breast cancer is called “inflammatory” because the breast often looks swollen and red, or inflamed. Inflammatory breast cancer is rare, accounting for 1 to 5 percent of all breast cancers diagnosed in the United States. Most inflammatory breast cancers are invasive ductal carcinomas, which means they developed from cells that line the milk ducts of the breast and then spread beyond the ducts. Inflammatory breast cancer progresses rapidly, often in a matter of weeks or months. At diagnosis, inflammatory breast cancer is either stage III or IV disease, depending on whether cancer cells have spread only to nearby lymph nodes or to other tissues as well. Inflammatory breast cancer is generally treated first with systemic chemotherapy to help shrink the tumor, then with surgery to remove the tumor, followed by radiation therapy. This approach to treatment is called a multimodal approach. Studies have found that women with inflammatory breast cancer who are treated with a multimodal approach have better responses to therapy and longer survival. Because inflammatory breast cancer usually develops quickly and spreads aggressively to other parts of the body, women diagnosed with this disease, in general, do not survive as long as women diagnosed with other types of breast cancer. See, www.cancer.gov/types/breast/ibc-fact-sheet.

### Dose and Administration

The compositions, as described herein, are administered in effective amounts. The effective amount will depend upon the mode of administration, the particular condition being treated and the desired outcome. It will also depend upon, as discussed herein, the stage of the condition, the age and physical condition of the subject, the nature of concurrent therapy, if any, and like factors well known to the medical practitioner. For therapeutic applications, it is that amount sufficient to achieve a medically desirable result.

The anti-cancer agent may be administered by any appropriate method. Dosage, treatment protocol, and routes of administration for anti-cancer agents, including chemotherapeutic agents, radiotherapeutic agents, and anti-cancer vaccines, are known in the art and/or within the ability of a skilled clinician to determine, based on the type of treatment, type of cancer, etc.

In one aspect of the invention, the modified immune cells are administered after the period of time of administration of an anti-fugetactic agent. In one embodiment, the modified immune cells administered during a period of time wherein the fugetactic effect of the cancer cells/tumor is attenuated by the anti-fugetactic agent. The length of time and modes of administration of the modified immune cells will vary, depending on the immune cells, type of tumor being treated, condition of the patient, and the like. Determination of such parameters is within the capability of the skilled clinician.

A variety of administration routes are available. The methods of the invention, generally speaking may be practiced using any mode of administration that is medically acceptable, meaning any mode that produces effective levels of the active compounds without causing clinically unacceptable adverse effects.

Modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

### Methods of Treatment

In one aspect is provided methods for treating a patient having a tumor which expresses high levels of CXCL12 wherein said patient is administered an effective amount of modified immune overexpressing CXCR7 receptors on the outer cell surface. In another aspect of this invention is provided methods for treating a patient having a tumor which expresses high levels of CXCL12 wherein said patient is administered an effective amount of modified immune having no or substantially no CXCR4 receptors on the outer cell surface. In yet another aspect of this invention is provided methods the invention relates to an ex vivo modified immune cell modified to overexpress CXCR7 receptors and modified to have no or substantially no CXCR4 receptors on an cell outer surface of the modified immune cell. In one embodiment, the modified immune cells are administered in combination with at least one additional anti-fugetactic agent.

In one aspect, this invention relates to evading the fugetactic activity of tumor cells when delivered to a patient. Without being bound be theory, it is believed that the modified immune cells as described herein can act as a decoy to bind and degrade the CXCL12-induced fugetactic wall in order to allow immune cells to detect and destroy tumor cells. In addition, it is believed, without being bound by theory, that the modified immune cells as described herein can bypass the fugetactic wall created by high levels of CXCL12 in the surrounding tumor microenvironment to reach and kill the tumor cells and kill the tumor cells.

In one embodiment, the modified immune cells and anti-fugetactic agent are administered sequentially. In another embodiment, the modified immune cells and anti-fugetactic agent are administered simultaneously. In one embodiment, the modified immune cells administered after the period of time of administration of an anti-fugetactic agent. In one embodiment, the modified immune cells are administered during a period of time when the fugetactic effect is attenuated.

In one embodiment, the chemokine is CXCL12. In one embodiment, the cancer cell is a solid tumor cell. In one embodiment, the cancer cell is a leukemia cell. In one embodiment, the modified immune cells are administered to the patient within about 3 days of administering an anti-fugetactic agent to the patient. In one embodiment, the modified immune cells are administered within about 1 day of administering an anti-fugetactic agent to the patient.

In one aspect, this invention relates to a method for treating a solid tumor in a mammal which tumor expresses CXCL12 at a concentration sufficient to produce a fugetactic effect, the method comprising administering to said mammal an effective amount of modified immune cells for a sufficient period of time so as to evade said fugetactic effect. In one embodiment, the cancer cell is a solid tumor cell. In one embodiment, the cancer cell is a leukemia cell. In one embodiment, the modified immune cells are administered within about 3 days of completion of administration of an anti-fugetactic agent. In one embodiment, the modified immune cells are administered within about 1 day of completion of administration of an anti-fugetactic agent.

In one embodiment, the immune cells are administered systemically to the patient. In another embodiment, the immune cells are administered directly to the tumor or tumor locally, which without limitation can include into the tumor microenvironment.

In one embodiment, the immune cells are administered using a catheter, a microcatheter, or are injected or implanted proximal to or within the tumor.

The modified immune cells of the present invention can be administered to a patient by absolute number of cells, for example, the patient can be administered from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells per injection, or any ranges between, end points inclusive.

In other embodiments, the amount of modified immune cells administered to a patient may calculated by kg of body weight. In general, such amount is at least 1×10³ modified immune cells per kg of body weight and most generally need not be more than 1×10⁹ modified immune cells/kg, e.g., 1×10³ cells/kg, 1×10⁴ cells/kg, 1×10⁵ cells/kg, 1×10⁶ cells/kg, 1×10⁷ cells/kg, 1×10⁸ cells/kg, 1×10⁹ cells/kg per injection, or any ranges between, end points inclusive.

The modified immune cells can be administered once to a patient who has or is suspected of having a cancer or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive. In some embodiments, the anti-fugetactic agent is delivered for at least one day prior to administration of the modified immune cells.

### Kit of Parts

This invention further relates to a kit of parts comprising modified immune cells and optionally an anti-fugetactic agent and/or an anti-cancer agent. In one embodiment, the kit of parts comprises a first container comprising the modified immune cells and an additional container or containers comprising an anti-fugetactic agent and an anti-cancer agent. In one embodiment, the kit of parts comprises a first set of prefilled syringes comprising modified immune cells and optionally additional sets of prefilled syringes containing an injectable form of an anti-fugetactic agent and/or an anti-cancer agent. In one embodiment, the kit of parts further comprises instructions in a readable medium for dosing and/or administration of the modified immune cells and optional anti-fugetactic agent and an anti-cancer agent.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

A Chimeric receptor gene is designed and created based on the contemplated tumor and its associated antigen. The target tumor-associated antigen is cloned as single chain Fc (“scFv”) molecule in to the fUSE5 vector phage DNA. After immunoscreening with antibody specific binding phage (such as GD2-binding phages), the selected scFv clone is ligated into pRSV-γ to assemble chimeric γ chain receptor. The transmembrane and cytoplasmic portions of human ζ chain are amplified from pGEM3zζ. SFG retroviral vector is used to constructed all the chimeric genes together by subcloned into its BamHI and NcoI sites.

Phoenix Eco cell line (American Type Culture Collection SD3444) is transiently transfected with constructed retroviral vector for production of recombinant retrovirus with CAR genes. The collected fresh retroviral supernatants are applied to infect PG13 cells (gibbon ape leukemia virus pseudotyping packaging cell line; American Type Culture Collection CRL-10686) for generation of a clinical application of self-inactivating retroviral vectors.

T cells are isolated using the EASYSEP™ Mouse T CellsIsolation Kit (STEMCELL™ Technologies) following the manufacturer's protocol. The isolated T cells are cultured in vitro, activated and expanded. The activated T cells are transfected with the clinical application of self-inactivating retroviral vectors.

siRNA sequence is designed as shRNA based on the CXCR4 sequence. The sequence of the shRNA is then cloned into plasmid pGCL-GFP, which encodes an HIV-derived lentiviral vector containing multiple cloning sites for insertion of shRNA. Virus is amplified using well-known techniques. The T cells are infected with the prepared recombinant lentivirus vector.

Mice are injected with tumor cells via subcutaneous injection to form a tumor that expresses high levels of CXCL12. Once the tumor is formed, the mice are injected (subcutaneous in the same flank as the tumor) with AMD3100 or vehicle, once a day for 5 days.

One to three days after the final dose of AMD3100, mice are injected via intravenous injection with 5×10⁶ T cells modified to express a CAR and have reduced CXCR4 receptors (CXCR4^{low} T cells) on their cell surface or unmodified T cells 18 hours prior to assay of tumor growth. Tumor growth in mice is delayed in mice treated with the CXCR4^{low} T cells, but continues in mice treated with unmodified T cells. It is contemplated that treatment with AMD3100 prior to treatment with CXCR4^{low} T cells will have a synergistic effect, such that the co-treatment results in a delay in tumor growth that is longer than CXCR4^{low} T cells alone.

### Example 2

T cells are isolated using the EASYSEP™ Mouse T Cell Isolation Kit (STEMCELL™ Technologies) following the manufacturer's protocol. Adenoviruses are constructed to contain the coding regions of CXCR7. To infect the isolated T cells, 25 multiplicity of infection (MOI) of the adenovirus are incubated for 24 hours and then the medium was replaced with fresh medium.

Mice are injected with tumor cells (subcutaneous injection) form a tumor that expresses high levels of CXCL12. Once the tumor has formed, the mice are injected (subcutaneous in the same flank as the tumor) with AMD3100 or vehicle, once a day for 5 days.

One to three days after the final dose of AMD3100, mice are injected via intravenous injection with 5×10⁶ CXCR7-modified T cells (overexpressing CXCR7 on their cell surface) or unmodified T cells 18 hours prior to assay of tumor growth. Tumor growth in mice is delayed in mice treated with the CXCR7-modified T cells, but continues in mice treated with unmodified T cells. It is contemplated that treatment with AMD3100 prior to treatment with CXCR7-modified T cells will have a synergistic effect, such that the co-treatment results in a delay in tumor growth that is longer than CXCR7-modified T cells alone.

### Example 3

T cells are isolated using the EASYSEP™ Mouse T Cell Isolation Kit (STEMCELL™ Technologies) following the manufacturer's protocol and divided into two samples. To infect the first sample of isolated T cells, 25 multiplicity of infection (MOI) of the adenovirus constructed to contain the coding regions of CXCR7 are incubated for 24 hours and then the medium was replaced with fresh medium. The second sample of isolated T cells are transfected with a shCXCR4 knockdown lentiviral vector. The first and second sample are then combined to be composition containing 2.5×10⁶ CXCR7-modified T cells (overexpressing CXCR7 on the cell surface) and 2.5×10⁶ CXCR4^{low}-modified T cells (reduced CXCR4 on the cell surface).

Mice are injected with tumor cells (subcutaneous injection) form a tumor that expresses high levels of CXCL12. Once the tumor has formed, the mice are injected (subcutaneous in the same flank as the tumor) with AMD3100 or vehicle, once a day for 5 days.

One to three days after the final dose of AMD3100, mice are injected via intravenous injection with 5×10⁶ T cell composition described above containing half of CXCR7-modified T cells and half of CXCR4^{low}-modified T cells or unmodified T cells 18 hours prior to assay of tumor growth. Tumor growth in mice is delayed in the mice treated with the modified T cell composition, but continues in mice treated with unmodified T cells. It is contemplated that treatment with AMD3100 prior to treatment with CXCR7-modified T cells and CXCR4^{low}-modified T cells will have a synergistic effect, such that the co-treatment results in a delay in tumor growth that is longer than CXCR7-modified T cells alone or CXCR4^{low}-modified T cells.

All references cited herein are hereby incorporated by reference in their entireties.

1. An ex vivo modified immune cell which comprises no or substantially no CXCR4 receptors on an outer cell surface of the modified immune cell.

2. The cell of claim 1, further comprising a tumor cell homing receptor on the cell surface.

3-5. (canceled)

6. The cell of claim 1, wherein the modified immune cell is a T cell, a B cell, or a natural killer (“NK”) cell.

7. (canceled)

8. The cell claim 2, wherein the tumor cell homing receptor is a chimeric antigen receptor (“CAR”), an Fc receptor, or a combination thereof.

9. The cell of claim 8, wherein the CAR targets a tumor-associated antigen.

10. (canceled)

11-24. (canceled)

25. A pharmaceutical composition comprising an effective amount of the modified immune cell of claim 1 and one or more pharmaceutically acceptable excipients.

26. (canceled)

27. An ex vivo modified immune cell modified to overexpress CXCR7 receptors on an outer cell surface of the modified immune cell.

28-30. (canceled)

31. The cell of claim 27, wherein the immune cell is a T-cell, B-cell, or natural killer cell.

32. The cell of claim 27, wherein the immune cell is further modified to express a tumor cell homing receptor on the surface of the immune cell.

33. The cell of claim 32, wherein the tumor cell homing receptor is a chimeric antigen receptor, an Fc receptor, or combinations thereof.

34. The cell of claim 33, wherein the chimeric antigen receptor targets a cancer-associated antigen.

35-46. (canceled)

47. A pharmaceutical composition comprising an effective amount of the modified immune cell of claim 27 and one or more pharmaceutically acceptable excipients.

48. An ex vivo modified immune cell modified to overexpress CXCR7 receptors and modified to have no or substantially no CXCR4 receptors on an outer cell surface of the modified immune cell.

49-52. (canceled)

53. The cell of claim 48, wherein the immune cell is a T-cell, B-cell, or natural killer cell.

54. The cell of claim 48, wherein the immune cell is further modified to express a tumor cell homing receptor on the surface of the immune cell.

55. The cell of claim 54, wherein the tumor cell homing receptor is a chimeric antigen receptor, an Fc receptor, or combinations thereof.

56. The cell of claim 55, wherein the chimeric antigen receptor targets a cancer-associated antigen.

57-71. (canceled)

72. A pharmaceutical composition comprising an effective amount of the modified immune cell of claim 48 and one or more pharmaceutically acceptable excipients.

73. (canceled)

74. A method for treating a patient having a tumor which expresses CXCL12 wherein said patient is administered an effective amount of modified immune cells or compositions of claim 1.

75-77. (canceled)

78. The method of claim 74, wherein the immune cells are administered in combination with an anti-fugetactic agent.

79-86. (canceled)
